(19) United States
(12) Patent Application Publication
(10) Pub. No.: US 2008/0063737 A1
(43) Pub. Date: Mar. 13, 2008

(54) Cosmetic-dermatological topical preparation and manufacturing process thereof

(76) Inventors: Maria Rosaria Brunese, Van Nuys, CA (US); Julie Brunese, Van Nuys, CA (US); Roberto Brunese, Van Nuys, CA (US)

Correspondence Address: Maria Rosaria Brunese, 6838 Whitaker Ave, Van Nuys, CA, 91406, US

(21) Appl. No.: 11/899,793

(22) Filed: Sep. 6, 2007

Related U.S. Application Data
(60) Provisional application No. 60/842,806, filed on Sep. 7, 2006.

(51) Int. Cl. A61K 36/886; A61P 17/02; A61P 17/10

(52) U.S. Cl. 424/744

(57) ABSTRACT

A cosmetic-dermatological topical preparation useful in the treatment of cosmetic-dermatological conditions such as striae, commonly known as stretch marks, acne, age spots, burn marks and in general in a variety of skin marks or imperfections. The core of the effectiveness of the preparation lies not only in the specific ingredients composition, dosage and physical form—gel lotion or oil—, but also in the way the preparation is manufactured.

### RELATED CASES

This application is the patent application of corresponding Provisional Patent Application No. 60/842,806 entitled “Cosmetic-Dermatological Topical Preparation and Manufacturing Process Thereof” filed on Sep. 7, 2006.

### FIELD OF THE INVENTION

The invention consists of a cosmetic-dermatological preparation useful in the treatment of cosmetic-dermatological conditions such as striae commonly known as stretch marks, acne and in general in the treatment of a variety of skin marks or imperfections.

### BACKGROUND OF THE INVENTION

Numerous topical preparations, pharmacological and cosmetic, are presently available in the market for the treatment, reduction, alleviation or even cure of striae, acneic eruptions, comedons and scars, and for the treatment of a variety of skin imperfections generally grouped under the name of blemishes.

Most of these preparations are marginally effective. Acne, in the eruption phase, is more amenable to treatment than the striae. Amelioration of acneic eruptions and comedons can be achieved by a variety of compounds including the well-known prescription drug Accutane.

With regard to the striae, instead, when they are formed, there is very little or nothing that can be done for their elimination or even reduction.

Despite some of the preparations on the market advertise successful treatment of striae, it is applicants opinion that actually there is not a single topical preparation which has shown to be consistently effective in the treatment of striae.

A variety of ingredients have been proposed and utilized for the treatment of the above conditions. Applicants believe that no single known ingredient has been capable of resolving the cosmetic problems associated with the above conditions.

### BRIEF SUMMARY OF THE INVENTION

Applicants invention consists of a topical cosmetic-dermatological preparation such as a lotion, cream, ointment and the likes, comprising a variety of components.

The cosmetic-dermatological preparation includes vitamins in a gel, lotion and oil form, each form alone or in combination, a phytochemical preparation in gel, lotion and oil form, each form alone or in combination, an antiseptic compound and an antibiotic ointment.

Applicants believe that the benefits achieved with the use of the proposed cosmetic-dermatological preparation are not only due to the synergistic association of the ingredients alone or in combinationsbut it is also the specific manufacturing process that makes the topical preparation effective.

The topical preparation if used on a consistent basis has shown the ability of improving the cosmetic appearance of striae, acne at different stage of evolution, age spots, burn marks and a variety of skin blemishes. Applicants invention does not claim to be the cure for such cosmetic-dermatological conditions. Applicants simply claim that it is the repeated empirical use of the preparation that generally improves the macroscopic cosmetic appearance of the above conditions.

Microscopic histochemical changes, however, must play a role in the improved cosmetic post application appearance of the above conditions. Applicants cosmetic-dermatological preparation indeed does not mask, camouflage or disguise imperfections. It is not a cosmetic make up for those conditions.

It is applicants belief that the cosmetic-dermatological preparation subject of the present invention acts at the core of the above conditions inducing dermatological changes, which result into an improved macroscopic appearance of the skin marks or imperfections in general.

### DETAILED DESCRIPTION OF THE INVENTION

The cosmetic-dermatological preparation subject of the present invention include the following ingredients or components or means:

Aloe Vera in a Lotion form such as:

Allantoin, Aloe Vera, Cetyl Alcohol, Chlorophyll, Collagen, di-Alpha Tocopheryl Acetate (Vitamin E), DL Panthenol, Fragrance, Glyceryl Stearate SE, Isopropyl Myristate, Methylparaben, Propylene Glycol, Propylparaben, Purified Soft Water, Safflower oil, Sodium PCA, Stearic Acid, Urea.

Vitamin E in a Lotion form such as:

Allantoin, Cetyl Alcohol, Cholecalciferol (Vitamin D), di-Alpha Tocopheryl Acetate (Vitamin E), DL Panthenol, Fragrance, Glyceryl Stearate SE, Isopropyl Myristate Polyethylene Glycol, Methylparaben, Propylparaben, Purified Soft Water, Retinyl A Palmitate, Safflower oil, Sodium PCA, Stearic Acid, Urea, Vegetable Oil, Wheat Germ Oil.

Vitamin E&C in a Lotion form such as:

Ascorbyl Palmitate (Vitamin C), Cetearyl Alcohol and Ceteareth-20, Cyclomethicone, Dimethicone, Methyl Gluseth, Octyl Palmitate, Panthenol (Vitamin B), Polysorbate 60, Propylene Glycol/Diazolidynl, Sorbitan Stearate, Tocopheryl Acetate (Vitamin E), Urea/Methylparaben/Propylparaben.

Aloe Vera in a Gel form such as:

Aloe Vera Gel, Carbomer & Triethanolamine, Panthenol, Propylene Glycol/Diazolidynl, Purified Soft Water, Urea/Methylparaben/Propylparaben.

Vitamin A in a Gel form such as:

Carbomer, d-Alpha Tocopheryl Succinate, Fragrance, Methylparaben, Polysorbate 80, Potassium Sorbate, Purified Soft Water, Retinyl A Palmitate.

Vitamin E in a Gel form such as:

Annatto, Carbomer, d-Alpha Tocopheryl Succinate, Fragrance, Methylparaben, Polyethylene Glycol, Potassium Sorbate, Purified Soft Water.

Vitamin E in a Oil form such as:

Safflower oil, Tocopheryl Acetate (Vitamin E).

Vitamin E&K in a Oil form such as:

Phytonadione (Vitamin K), Safflower Oil, Tocopheryl Acetate (Vitamin E).

Benzoyl Peroxide in a preparation such as:

Benzoyl Peroxide 5%, Carbomer 940, Dimethicone, Disodium Lauryl Sulfosuccinate, Edetate Disodium, Glycerin, Hydrated Silica, Methylparaben, Poloxamer 182, Purified Soft Water, Sodium Hydroxide.

Double Antibiotic Ointment:

Bacitracin Zinc, Polymyxin B Sulfate, White Petrolatum.

The cosmetic dermatological preparation can be made of the above listed components/ingredients/means in gel form and/or lotion form and/or oil form components all in the same form i.e. gel form or lotion form or oil form or in various forms combination.

Applicants' cosmetic empirical use suggests that the combination of components in lotion, gel and oil form in a mixture is the most effective. The three principal ingredients are:

Aloe vera in gel form and/or lotion form and/or oil form, Vitamin E in gel form and/or lotion form and/or oil form and the Antibiotic ointment.

As indicated in the Brief Summary of the Invention, it is Applicants belief that the effectiveness of the preparation is also, and may be largely, due to the way it is manufactured.

Here below the manufacturing process in details:

In a first container mix the following ingredients in the following quantities:

Vitamin E oil, 30 ml—Double Antibiotic Ointment, 30 ml—Benzoyl Peroxide 5%, 20 ml—Aloe Vera Gel, 60 ml—Vitamin E Gel, 60 ml—Vitamin A Gel, 90 ml—Vitamin E & K oil.

Use a double boiler type of equipment to heat the above mixture and melt the ingredients together.

In a second container mix, at room temperature, the following ingredients in the following quantities:

Aloe Vera Lotion, 200 ml—Vitamin E Lotion, 200 ml—Vitamin E & C Lotion, 25 ml.

Pour the content of the first container, slightly cooled, into the second container. Stir until the ingredients of both containers are well mixed and dispersed uniformly.

Use.

Applicants have empirically used the preparation in at least twenty different volunteer subjects.

The reductions of striae were dramatic in at least five subjects. In another five subjects the grade of reduction was not as dramatic but still evident after daily application for at least thirty days.

With regards to acne the preparation has been used in five subjects with different degree of success at different stages, the eruption, comedon and scar stages.

The topical preparation has been also used for a variety of skin marks and or imperfections with different degree of success.

In all subjects the appearance of the blemishes was improved after daily treatment for at least thirty days to the point that in all cases those marks were definitely less visible, less macroscopically noticeable.

What we claim is:

1. A cosmetic dermatological topical preparation for treatment of stretch marks acne and skin blemishes in general comprising:
an aloe vera ingredient, a vitamin E ingredient and an antibiotic ointment ingredient.

2. The cosmetic dermatological topical preparation of claim 1 further comprising a vitamin A ingredient.

3. The cosmetic dermatological topical preparation of claim 1 further comprising vitamin C ingredient.

4. The cosmetic dermatological topical preparation of claim 1 further comprising vitamin K ingredient.

5. The cosmetic dermatological topical preparation of claim 1 fuirther comprising a Benzoyl Peroxide ingredient.

6. The cosmetic dermatological topical preparation of claim 1 wherein the aloe vera ingredient is in a lotion form.

7. The cosmetic dermatological topical preparation of claim 1 wherein the aloe vera ingredient is in a gel form.

8. The cosmetic dermatological topical preparation of claim 1 wherein the aloe vera ingredient is in a oil form.

9. The cosmetic dermatological topical preparation of claim 1 wherein the vitamin E ingredient is in a lotion form.

10. The cosmetic dermatological topical preparation of claim 1 wherein Vitamin E ingredient is in a gel form.

11. The cosmetic dermatological topical preparation of claim 1 wherein the vitamin E ingredient is in a oil form.

12. The cosmetic dermatological topical preparation of claim 1 wherein the vitamin E ingredient is in a oil form.

13. The cosmetic dermatological topical preparation of claim 1 wherein the antibiotic ingredient contain bacitracyn.

14. The cosmetic dermatological topical preparation of claim 1 wherein the antibiotic ingredient contain polysporin.

15. The cosmetic dermatological topical preparation of claim 1 wherein the aloe vera ingredient and the vitamin E ingredient are each in a gel, lotion and oil forms in variuos combinations.

16. A method of manufacturing a cosmetic dermatological preparation comprising the steps of:
1) mixing into a uniform mixture in a first container the following ingredients:
Vitamin E oil, Vitamin E gel, Double Antibiotic Ointment, Aloe Vera Gel, Vitamin A Gel, Vitamin E & K oil, Benzoyl Peroxide and
2) Heating the mixture of step 1 in a double boiler type of equipment to melt the ingredients together and
3) Mixing, at room temperature, in a second container the following ingredients:
Aloe Vera Lotion, Vitamin E Lotion, Vitamin E & C Lotion.
4) Pouring the content of the first container, slightly cooled, into the second container and
5) Stirring the mixture until the ingredients of the first and second container are well mixed and dispersed uniformly.
